(19) United States
(12) Patent Application Publication
(10) Pub. No.: US 2004/0067963 A1
(43) Pub. Date: Apr. 8, 2004

(54) Methods to prevent or ameliorate trauma-related psychological disorders

(75) Inventors: Nathan Andrew Shapira, (US); Serena M. Galen, (US); Mary Catherine Lessig, (US)

Correspondence Address: Frank C. Eisenschenk, Ph.D., Saliwanchik, Lloyd & Saliwanchik,, A Professional Association, 2421 N.W. 41st Street, Suite A-1, Gainesville, FL, 32606, US

(73) Assignee: University of Florida

(21) Appl. No.: 10/459,550

(22) Filed: Jun. 10, 2003

Related U.S. Application Data
(60) Provisional application No. 60/394,593, filed on Jul. 8, 2002.

(51) Int. Cl. A61K 31/4965; A61K 31/495; A61K 31/137

(52) U.S. Cl. 514/255.04; 514/649

(57) ABSTRACT

The subject invention provides methods for the treatment of ASD and/or PTSD comprising the administration of a dose of an SSRI taken soon after a trauma (for example, less than 48 hours and before the onset of ASD.

### CROSS-REFERENCE TO RELATED APPLICATION

This application claims the benefit of U.S. Provisional Application Serial No. 60/394,593, filed Jul. 8, 2002.

### BACKGROUND OF INVENTION

One in four people will experience a traumatic event—a natural disaster, a war, or violent abuse—at some time in their lives (LARKIN, M., “Can post-traumatic stress disorder be put on hold?”, The Lancet, 1999, 354:1008). About 15% of these people will develop post-traumatic stress disorder (PTSD), a condition with a lifetime prevalence in the general population between 1% and 9% and an even higher prevalence among particular subpopulations (e.g., a current incidence between 15% and 28% among Vietnam veterans) (MARTENYI, F., et al., “Fluoxetine Versus Placebo in Posttraumatic Stress Disorder”, J. Clin. Psychiatry, 2002, 63:199206).

PTSD is characterized by 3 specific groups of symptoms: intrusive behaviors (including flashbacks and intense physiologic distress), avoidance behaviors (including avoidance of reminders of the trauma and numbing of responsiveness), and hyperarousal (including insomnia and an exaggerated startle response); in order to make the diagnosis of PTSD, these symptoms must cause clinically significant impairment and continue for at least 30 days after the stressor (MARTENYI, F., et al., “Fluoxetine Versus Placebo in Posttraumatic Stress Disorder”, J. Clin. Psychiatry, 2002, 63:99-206; STEIN, D., et al., “Selective serotonin reuptake inhibitors in the treatment of post-traumatic stress disorder: a meta-analysis of randomized controlled trials”, Int. Clin. Psychopharmacol, 2000, 15 (suppl 2):S31-39). Acute stress disorder (ASD), with symptoms characteristic of PTSD for more than 2 days but less than 1 month in duration, has been shown in prospective studies to be a useful predictor of PTSD.

For example, 79 survivors of motor vehicle accidents who sustained mild traumatic brain injury were assessed for ASD within 1 month of the trauma; 63 and 50 of those initially assessed were evaluated for PTSD at 6 months and 2 years, respectively. ASD was diagnosed in 14% of the patients, and PTSD was diagnosed in 24% at 6 months post-trauma and 22% at 2 years post-trauma. Among the patients who participated in all three assessments, 78% and 80% of the subjects who met the criteria for ASD were diagnosed with PTSD at 6 months and 2 years, respectively. Of the total initial group, 73% of those diagnosed with ASD had PTSD at 2 years (HARVEY, A., et al., “Two-Year Prospective Evaluation of the Relationship Between Acute Stress Disorder and Posttraumatic Stress Disorder Following Mild Traumatic Brain Injury”, Am. J. Psychiatry, 2000, 157:626-28; HARVEY, A., et al., “The relationship between acute stress disorder and posttraumatic stress disorder: a 2-year prospective evaluation”, J. Consult Clin. Psychol., 1999, 67(6):985-8).

These numbers suggest that acute post-trauma interventions are lacking or unsuccessful in preventing or ameliorating chronic post-trauma psychiatric morbidity. A primary reason for this is that psychological resistance may be of considerable importance in the PTSD population, considering that researchers in the field of traumatic stress are frequently unsuccessful in achieving high response rates, that many subjects suffering from PTSD never seek help, and that dropouts from therapy are frequent (WEISAETH, L., “Acute post-traumatic stress: nonacceptance of early intervention”, J. Clin. Psychiatry, 2001, 62 Suppl, 17:35-40). In a significant proportion of the acutely distressed, the reluctance to seek help is motivated by the very symptoms that predicted PTSD; for primary and secondary prevention, early screening and outreach should be very active (WEISAETH, L., “Acute posttraumatic stress: nonacceptance of early intervention”, J. Clin. Psychiatry, 2001, 62 Suppl, 17:35-40).

Trauma is as diverse as its psychiatric manifestations and negative sequelae, which affect society as well as the individual. However, reliance on the categorical model of psychiatric disorders has led to neglected study of post-traumatic sequelae that do not necessarily meet full criteria for PTSD, but are nevertheless associated with substantial comorbidity, disability, and suicidality (MARSHALL, R., et al., “Comorbidity, Impairment, and Suicidality in Subthreshold PTSD”, Am. J Psychiatry, 2001, 158:1467-1473). Since the identification in 1980 of PTSD as a diagnosable syndrome, numerous studies have demonstrated a high degree of associated psychiatric comorbidity and disability, including work-related impairment, somatic complaints, lower quality of life, suicidality, medical illness, negative body image, impaired intimacy, increased burden to spouse or partner, social dysfunction, chronic pain, substance abuse, anxiety, and depression (MARSHALL, R., et al., “Comorbidity, Impairment, and Suicidality in Subthreshold PTSD”, Am. J. Psychiatry, 2001, 158:1467-1473; JACOBSEN, L., et al., “Substance Use Disorders in Patients With Posttraumatic Stress Disorder: A Review of the Literature”, Am. J. Psychiatry, 2001, 158:1184-1190; SHARP, T., et al., “Chronic pain and posttraumatic stress disorder: mutual maintenance?”, Clinical Psychology Review, 2001, 21:857-77; SCHNYDER, U., et al., “Prediction of Psychiatric Morbidity in Severely Injured Accident Victims at One-year Follow-up”, Am. J. Respir. Crit. Care Med., 2001, 164:653-56).

In a study of 9,358 subjects reporting PTSD symptoms on National Anxiety Disorders Screening Day 1997, regression analysis was used to determine that the rates of impairment, comorbid anxiety disorders, and comorbid major depressive disorder were 31.5%, 68.5%, and 90.7% higher, respectively, among the subjects with four PTSD symptoms than among the subjects with no PTSD symptoms (MARSHALL, R., et al., “Comorbidity, Impairment, and Suicidality in Subthreshold PTSD”, Am. J. Psychiatry, 2001, 158:1467-1473). Significantly, subthreshold PTSD was associated with a significantly higher risk for current suicidal ideation, similar to that of OCD and higher than that of panic disorder or social phobia, after controlling for the presence of major depressive disorder; more than three times as many individuals with full PTSD reported current suicidal ideation than those with no PTSD symptoms (MARSHALL, R., et al., “Comorbidity, Impairment, and Suicidality in Subthreshold PTSD”, Am. J. Psychiatry, 2001, 158:1467-1473).

In another study of psychiatric morbidity in 106 accident victims at one-year follow-up, 25.5% of patients showed some form of psychiatric morbidity (full or subsyndromal PTSD and/or anxiety and/or depression) (SCHNYDER, U., et al., “Prediction of Psychiatric Morbidity in Severely Injured Accident Victims at One-year Follow-up”, Am. J. Respir. Crit. Care Med., 2001, 164:653-56). Similarly, in another study of 1, 441 consecutive patients admitted to an ER after a motor vehicle accident and subsequently followed for post-trauma pathology at 3-month and 1-year follow-up, PTSD, phobic travel anxiety, general anxiety, and depression were reported by a third of the subjects at both 3 months and 1 year (MAYOU, R., et al., “Prediction of Psychological Outcomes One Year After a Motor Vehicle Accident”, Am. J Psychiatry, 2001, 158:1231-1238).

Numbers indicate that trauma has an enormous impact on the individual as well as society at large. In a case-comparison study of 102 high users of VA health services and 54 low users who were assessed for PTSD diagnosis and severity of symptoms, high users of health care were almost twice as likely as low users (27.5% vs. 14.8%) to meet diagnostic criteria for current PTSD (DEYKIN, E., et al., “Posttraumatic stress disorder and the use of health services”, Psychosom. Med., 2001, 63:835-41). A review of family practice literature revealed that, after spontaneous abortion, as many as 10% of women may have ASD and up to 1% may have PTSD (BOWLES, S., et al., “Acute and post-traumatic stress disorder after spontaneous abortion”, Am. Fam. Physician, 2000, 61:1689-96).

Psychiatric morbidity related to trauma must be addressed, but when and how? Pharmacotherapy options which have been proposed to treat rather than prevent chronic symptomatology include mood stabilizers, beta-blockers, alpha2-agonists, benzodiazepines, and antidepressants; however, a number of these agents have shown equivocal response rates, multiple doses must be taken, or adverse effects such as memory impairment for the traumatic event, which can hinder legal efforts (SUTHERLAND, S., et al., “Pharmacotherapy for Post-Traumatic Stress Disorder”, Psychiatric Clinics of North America, 1994, 17:409-23). While many questions remain about the differential response of different PTSD symptoms and various PTSD patients, the SSRIs appear useful in treating PTSD as well as its comorbid conditions with relatively few side effects (SUTHERLAND, S., et al., “Pharmacotherapy for Post-Traumatic Stress Disorder”, Psychiatric Clinics of North America, 1994, 17:409-23).

For example, in a recent double-blind, randomized, placebo-controlled study of 301 patients with combat-related PTSD, it was determined that by week 6 of treatment with fluoxetine at doses in the normal to upper range for the usual antidepressant doses, there was a statistically significant improvement from baseline in total Treatment Outcome PTSD rating scale score than for placebo (MARTENYI, F., et al., “Fluoxetine Versus Placebo in Posttraumatic Stress Disorder”, J. Clin. Psychiatry, 2002, 63:199-206). Similarly, significant superiority of sertraline compared with placebo was observed in a 12 week trial of 187 outpatients with PTSD who were primarily female victims of sexual assault (MARTENYI, F., et al., “Fluoxetine Versus Placebo in Posttraumatic Stress Disorder”, J. Clin. Psychiatry, 2002, 63:199-206).

It appears that serotonin dysregulation plays a role in much post-trauma symptomatology. For example, in a retrospective chart assessment after at least 1 month of medication in 72 patients with PTSD and comorbid depression, serotonergic antidepressants were associated with better outcomes than those of the noradrenergic type; response rates of greater than 30% were obtained for fluotexine, sertraline, imipramine, and phenelzine, while response rates of less than 10% were obtained with several agents including desipramine, amitriptyline, nortriptyline and bupropion (STEIN, D., et al., “Selective serotonin reuptake inhibitors in the treatment of post-traumatic stress disorder: a meta-analysis of randomized controlled trials”, Int. Clin. Psychopharmacol, 2000, 15 (suppl 2):S31-39). Although there is no direct evidence of serotoninergic function in PTSD, several observations from animal models suggest why SSRIs may be effective in its treatment. Low CSF levels of 5-HIAA have been associated with poor impulse control, which is a pre-eminent feature in PTSD (SUTHERLAND, S., et al., “Pharmacotherapy for Post-Traumatic Stress Disorder”, Psychiatric Clinics of North America, 1994, 17:409-23). Additionally, successful stress adaptation in animals is associated with increased central 5-HT function (SUTHERLAND, S., et al., “Pharmacotherapy for Post-Traumatic Stress Disorder”, Psychiatric Clinics of North America, 1994, 17:409-23). These observations suggest that serotonergic drugs many have particular efficacy in the treatment of several aspects of PTSD, especially the avoidance symptoms and perhaps also the impulsiveness associated with the disorder (SUTHERLAND, S., et al., “Pharmacotherapy for Post-Traumatic Stress Disorder”, Psychiatric Clinics of North America, 1994, 17:409-23).

It has also been observed that patients with PTSD have significantly lower heart rate variability (HRV) compared to controls, reflecting a basal autonomic state characterized by increased sympathetic and decreased parasympathetic tone, which would account for the hyperarousal symptoms characteristic of PTSD (COHEN, H., et al., “Normalization of Heart Rate Variability in Post-Traumatic Stress Disorder Patients Following Fluoxetine Treatment: Preliminary Results”, IMAJ, 2000, 2:296-300). This may be due to the fact that serotonin (including dopamine and other catecholamines) influence the heart via the CNS, or by the interactions between the serotonergic and noradrenergic systems (COHEN, H., et al., “Normalization of Heart Rate Variability in Post-Traumatic Stress Disorder Patients Following Fluoxetine Treatment: Preliminary Results”, IMAJ, 2000, 2:296-300). In a study of 16 patients with HRV parameters indicating autonomic dysregulation characteristic of PTSD, those treated with 20-60 mg of fluoxetine showed significantly higher HRV than controls, indicating that SSRIs may improve HRV parameters and autonomic dysregulation in post-trauma symptomatology (COHEN, H., et al., “Normalization of Heart Rate Variability in Post-Traumatic Stress Disorder Patients Following Fluoxetine Treatment: Preliminary Results”, IMAJ, 2000, 2:296-300).

Nevertheless, questions remain about the timing, dosing, and duration of SSRI treatment. Early improvement (i.e., 2-4 weeks) to an SSRI seems to predict response at week 12 (STEIN, D., et al., “Selective serotonin reuptake inhibitors in the treatment of post-traumatic stress disorder: a meta-analysis of randomized controlled trials”, Int. Clin. Psychopharmacol, 2000, 15 (suppl 2):S31-39). For example, in a 12-week placebo controlled study of fluoxetine (up to 60 mg/day) in PTSD in 53 civilians, fluotexine had significantly more effect on symptom severity than placebo by week 2-4, and was more effective on most measures, including disability improvement, by week 12; patients who failed to respond by week 4 were unlikely to respond by week 12 (STEIN, D., et al., “Selective serotonin reuptake inhibitors in the treatment of post-traumatic stress disorder: a meta-analysis of randomized controlled trials”, Int. Clin. Psychopharmacol, 2000, 15 (suppl 2):S31-39; DAVIDSON, J., et al., “Response characteristics to antidepressants and placebo in post-traumatic stress disorder”, Int. Clin. Psychopharmacol., 1997, 12:291-96). It may, therefore, take up to 12 weeks of treatment to determine accurately the extent of possible symptomatic benefit and, although standard doses of SSRIs can be effective in PTSD, many clinicians would suggest maximizing recommended doses if these are tolerated (STEIN, D., et al., “Selective serotonin reuptake inhibitors in the treatment of post-traumatic stress disorder: a meta-analysis of randomized controlled trials”, Int. Clin. Psychopharmacol, 2000, 15 (suppl 2):S31-39).

It is important to note that one preventive measure available to post-trauma victims involves psychological debriefing, which is a type of counseling developed during WWII aimed at enabling cognitive appraisal and emotional processing of traumatic experiences (KAPLAN, Z., et al., “A Review of Psychological Debriefing After Extreme Stress”, Psychiatric Services, 2001, 52:824-27). However, in some cases this technique results in more post-traumatic symptomatology. For example, in a sample of 243 traumatized police officers, a subgroup of debriefed officers was compared with non-debriefed internal and external control groups. No differences in psychological morbidity were found between the groups at pre-test, at 24 hours or at 6 months post-trauma. One week post-trauma, debriefed subjects exhibited significantly more PTSD symptomatology than non-debriefed subjects (CARLIER, I., et al., “The influence of occupational debriefing on post-traumatic stress symptomatology in traumatized police officers”, British Journal of Medical Psychology, 2000, 73:87-98).

### BRIEF SUMMARY

The subject invention provides methods for the treatment of ASD and/or PTSD comprising the administration of a dose of an SSRI taken soon after a trauma (for example, less than 48 hours and before the onset of ASD).

### DETAILED DISCLOSURE

The subject invention describes methods of preventing or ameliorating trauma related psychological disorders in an individual. The invention provides for the treatment of an individual, post trauma, with (for example, 0.1 mg to 500 mg) of one or more SSRI compounds. In some embodiments, the SSRI containing composition is administered after a traumatic event (e.g., such as 1 minute to 48 hours post-trauma). In other embodiments, a single dose (or multiple doses) of a SSRI containing composition is administered within one to 48 hours, 2 to 46 hours, 3 to 44 hours, 4 to 42 hours, 5 to 40 hours, 6 to 38 hours, 7 to 36 hours, 8 to 34 hours, 9 to 32 hours, 10 to 30 hours, 11 to 28 hours, 12 to 24 hours, 12 to 26 hours, or 10 to 12 hours of the trauma. The goal of the treatment regimen is to prevent or blunt the development of ASD or PTSD in the individual. Additionally, the subject method can be used to blunt, treat, ameliorate, or prevent other post-trauma psychiatric morbidity, including depression, dysthymia, bipolar disorder, panic disorder, OCD, GAD, SAD, specific phobia, substance abuse/dependence, chronic pain, somatoform disorders, eating disorders, personality disorders, impulse control disorders, sleep disorders, and dissociative disorders and additional doses of an SSRI (or combination of SSRI) can be administered for the treatment of such disorders, including ASD and PTSD).

Exemplary SSRI for use in the subject invention include paroxetine or fluoxetine, preferably long-acting or sustained release (such as once-weekly fluoxetine [Prozac Weekly™]). Other SSRI suitable for use in the practice of the instant invention include ZOLOFT (sertraline), LUVOX (fluvoxamine), escitalopram, and/or CELEXA (citalopram). As indicated supra, various combinations of [e.g., up to six (6)] SSRIs, salts, derivatives, or analogs thereof, can be used in the practice of the instant invention.

The administration of an SSRI would have the effect of blunting, treating, preventing, or ameliorating ASD and PTSD since receptor densities and patient responses can take weeks to change. For example, the sensitization of the receptors mediating 5-HT occurs with a time course (2-3 weeks) that is congruent with the delayed onset of action of these drugs in major depression (BLIER, P., et al., “Possible Serotonergic Mechanisms Underlying the Antidepressant and Anti-Obsessive-Compulsive Disorder Responses”, Biol. Psychiatry, 1998, 44:313-23). Thus, the subject invention provides for the administration of one or more SSRIs within one to two thousand eight hundred eighty minutes (48 hours) of a traumatic event. The aforementioned range of 1 to 2880 minutes is to be taken as including, and providing written description and support for, any range of time in half minute or one minute intervals between 1 and 2880.

The subject invention has human and veterinary application. In veterinary application, it is recognized that animals can be traumatized by various events, including capture, restraint for veterinary procedures, involvement in vehicular accidents, shipping, or exposure to new living conditions (e.g., transfer to new zoos or enclosures). For humans, an extreme traumatic stressor (traumatic event) typically involves direct personal experience of an event that involves: a) an actual or threatened death or serious injury, or other threat to one's physical integrity; b) witnessing an event that involves death, injury, or a threat to the physical integrity of another person; or c) learning about unexpected or violent death, serious harm, or threat of death or injury experienced by a family member or other close associate. Traumatic events that are experienced directly include, but are not limited to, spontaneous abortion, military combat, violent personal assault (sexual assault, physical attack, robbery, mugging), being kidnapped, being taken hostage, terrorist attack, torture, incarceration as a prisoner of war or in a concentration camp, natural or manmade disasters, automobile accidents (including, but not limited severe automobile accidents), or being diagnosed with a life-threatening illness. For children, sexually traumatic events may include developmentally inappropriate sexual experiences without threatened or acutal violence or injury. Witnessed events include, but are not limited to, observing the serious injury or unnatural death of another person due to violent assault, accident, war, or disaster or unexpectedly witnessing a dead body or body parts. Events experienced by others that are learned about include, but are not limited to, violent personal assault, serious accident, or serious injury experienced by a family member or a close friend; learning about the sudden, unexpected death of a family member or a close friend; or leaning that one's child has a life-threatening disease.

The term “individual” includes animals of avian, mammalian, or reptilian origin. Mammalian species which benefit from the disclosed methods include, and are not limited to, apes, chimpanzees, orangutans, humans, monkeys; domesticated animals [(pets) such as dogs, cats, guinea pigs, hamsters, Vietnamese pot-bellied pigs, rabbits, and ferrets]; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, giant pandas, hyena, seals, sea lions, and elephant seals. Reptiles include, and are not limited to, alligators, crocodiles, turtles, tortoises, snakes, iguanas, and/or other lizards. Avian species include, and are not limited to, chickens, turkeys, pigeons, quail, parrots, macaws, dove, Guinea hens, lovebirds, parakeets, flamingos, eagles, hawks, falcons, condor, ostriches, peacocks, ducks, and swans.

Preferably, the SSRI are provided in unit dosage form and in a form adapted for use in the medical or veterinary fields. SSRI can be formulated as a pharmaceutically acceptable salt thereof may be formulated for administration by any route, and examples are oral, rectal, topical, parenteral, intravenous or intramuscular administration.

The compositions may, for example, be in the form of tablets, capsules, sachets, vials, powders, granules, lozenges, reconstitutable powders, or liquid preparations, for example solutions or suspensions, or suppositories. The compositions, for example those suitable for oral administration, may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycerine; tabletting lubricants, for example magnesium stearate; disintegrants, for example starch, polyvinylpyrrolidone, sodium starch glycollate or microcrystalline cellulose: or pharmaceutically acceptable setting agents such as sodium lauryl sulfate.

Solid compositions may be obtained by conventional methods of blending, filling, tabletting or the like. Repeated blending operations may be used to distribute paroxetine or a salt thereof throughout those compositions employing large quantities of fillers. When the composition is in the form of a tablet, powder, or lozenge, any carrier suitable for formulating solid pharmaceutical compositions may be used, examples being magnesium stearate, starch, glucose, lactose, sucrose, rice flour and chalk. Tablets may be coated according to methods well known in normal pharmaceutical practice, in particular with an enteric coating. The composition may also be in the form of an ingestible capsule.

Compositions for oral administration as liquids may be in the form of, for example, emulsions, syrups, or elixirs, or may be presented as a dry product for reconstitution with water or other suitable vehicle before use. Such liquid compositions may contain conventional additives such as suspending agents, for example sorbitol, syrup, methyl cellulose, gelatin, hydroxyethylcellulose, carboxymethylcellulose, aluminum stearate gel, hydrogenated edible fats; emulsifing agents, for example lecithin, sorbitan monooleate, or acacia; aqueous or non-aqueous vehicles, which include edible oils, for example almond oil, fractionated coconut oil, oily esters, for example esters of glycerine, or propylene glycol, or ethyl alcohol, glycerine, water or normal saline; preservatives, for example methyl or propyl p-hydroxybenzoate or sorbic acid; and if desired conventional flavoring or coloring agents.

As mentioned hereinbefore, the effective dose of the SSRI or pharmaceutically acceptable salt, derivative, or analog thereof depends on the severity of the disorders, the condition of the patient and on the route of administration. A unit dose will generally contain from 0.1 to 500 mg or 0.1 to 1000 mg and can contain from 5 to 500 mg, in particular 10-500, 20-500, 30-500, 40-500, 50-500, 100-500, 150-500, 200-500, 250-500, 300-500, 350-500, 400-500, 450-500 mg. Other embodiments provide for the administration of between 0.1 to 100 mg; 1 to 100 mg; 10 to 100 mg; 30-80 mg; 40-70 mg; 50-60 mg; or 20 and 90 mg of SSRI compositions to the individual. The composition may be administered once. Alternatively, the composition can be administered more than one time a day for example 2, 3 or 4 times daily.

In various embodiments, the subject invention provides for the administration of a single dose of an SSRI to an individual within 48 hours of a traumatic event for treating, blunting, preventing, or ameliorating trauma-related psychological disorders. Other embodiments provide for the administration of a single SSRI composition within 48 hours of a traumatic event followed by the administration of additional doses of SSRI containing compositions.

### EXAMPLE 1

### Animal Testing

In animals, stress leading to trauma has been exhibited through the use of the Acoustic Startle Chamber (ASC). An animal, e.g., a mouse, is placed in the chamber of the ACS and a series of pulses and tones are emitted. The level of startle is measured by a platform on the floor of the chamber (DAVIS, M., “Neurochemical modulation of sensory-motor reactivity: acoustic and tactile startle reflexes”, Neurosci Biobehav Rev, 1980, Summer, 4(2) 241-63). Levels of corticosterone (CORT) rise in the blood of the animal. CORT has been shown to be representative of the level of anxiety an animal is feeling following the introduction of a stressor (HENNESSY, M., et al., “Plasma corticosterone concentrations sensitively reflect levels of stimulus intensity in the rat”, Physiol Behav, 1979, 22:821-825).

There are several testing paradigms used to measure levels of anxiety in animals following the introduction of a stressor, including the Elevated Plus Maze (EPM) (LISTER, R., “The use of a plus-maze to measure anxiety in the mouse”, Psychopharmacology (Berl), 1987, 92:180-185); the Open Field Test (CHOLERIS, E., et al., “A detailed ethological analysis of the mouse open field test: effects of diazepam, chlordiazepoxide and an extremely low frequency pulsed magnetic field”, Neurosci Biobehav Rev, 2001, 25:235-260); and the reaction to Predatory Odor (ROY, V., et al., “Environmental enrichment of BALB/c mice: Effects in classical tests of anxiety and exposure to predatory odor”, Physiol Behav, 2001, 74:313-320).

The EPM consists of a central platform and 4 arms that radiate out from the central platform, mounted on a pedestal base that is between 24 and 38 inches above the ground, constructed of dark Plexiglas. Two arms ate open and two arms are enclosed with clear Plexiglas forming a box to shelter the animal. The amount of time an animal spends on the open arms of the maze has been shown to correlate with lowered levels of anxiety (LISTER, R., “The use of a plus-maze to measure anxiety in the mouse”, Psychopharmacology (Berl), 1987, 92:180-85). Experiments using anxiolytics have been shown to increase the amount of time an animal will spend on the open arms of maze.

In the Open Field Test (OFT), a sheltered start box is attached to an open arena typically divided into zones. Although thought to be a “standardized test”, an OFT minimally needs to encompass an enclosed arena for animal activity to be monitored (CHOLERIS, E., et al., “A detailed ethological analysis of the mouse open field test: effects of diazepam, chlordiazepoxide and an extremely low frequency pulsed magnetic field”, Neurosci Biobehav Rev, 2001, 25:235-260). The center zone of the open arena tends to be the area that an animal is least likely to spend time due to its desire to avoid predation, although the introduction of an anxiolytic medication or similar agent can enhance the amount of time an animal spends away from the edges of the enclosed open arena. Predatory Odor tests involve the introduction of a smell that invokes predator-prey responses, i.e., following a session in the ACS introducing cat feces in the home cage of a mouse to increase stress levels (ROY, V., et al., “Environmental enrichment of BALB/c mice: Effects in classical tests of anxiety and exposure to predatory odor”, Physiol Behav, 2001, 74:313-320).

Any of these tests, the EPM, OFT, or Predatory Odor, would be administered following a period in the ACS and after an acute administration of the desired pharmacologic agent (i.e., once weekly fluoxetine). In human volunteers, individuals, following a traumatic event (e.g., a car accident), would be administered an acute dose (for example, in less than 12, 24, 36, or 48 hours after the traumatic event) of the desired pharmacologic agent similar to the described animal method above vs. placebo. Standard clinical follow-up will determine the rate of ASD and other trauma-related disorders such as PTSD.

### EXAMPLE 2

Currently there are no approved or accepted treatments for preventing or ameliorating psychological disorders directly or in part related to trauma. No SSRIs have been formally tested for either preventing acute stress disorder (ASD) (and potentially lowering the later occurrence of PTSD and related disorders) or ameliorating the symptoms of acute stress disorder. Due to escitalopram's excellent tolerability and apparent quick response, it serves as an ideal compound for use in the claimed methods because of its potential to significantly reduce the extraordinary financial and social burden of these disorders.

In this aspect of the invention, escitalopram (and/or salts, derivatives, or analogs thereof), alone, or incombination with other SSRI compounds (and salts, derivatives, or analogs thereof), is administered within 6 hours of trauma to prevent, treat, blunt, or ameliorate the development of ASD/PTSD and related disorders such as other anxiety disorders (or other disorders as provided in paragraph 16, supra) in individuals. For example, individuals who have been in a car accident and who are medically cleared by an ER would be enrolled in a double-blind placebo-controlled administration of 10 mg escitalopram (either alone or in combination with other SSRI compounds) as close to the car accident as possible (within 6 hours) followed by 10 mg within the next day and the last dose of 10 mg within the day after that (2 days post trauma) with the goal of 30 mg administered within 48 hours. In order to maximize the speed of absorption of the first dose, liquid escitalopram can be utilized for the first dose followed by pills for the subsequent dosages in one embodiment. Subjects would be followed up on day 3 after the accident and then at weeks 1, 2, 4, and 8 after the accident. Optimally, the research coordinator at week 8 would be unaware of whether the subject developed ASD in the first month of the study.

Evaluations can include a MINI Neuropsychiatric Interview (baseline and week 8); the Acute Stress Disorder Structured Interview which includes a total score to evaluate severity (day 3 and weeks 1,2,4); the Acute Stress Disorder Scale (day 3 and weeks 1 and 2); the Stanford Acute Stress Reaction Questionnaire (day 3 and weeks 1,2,4), the Hamilton Depression Rating Scale (all visits); the Impact of Events Scale and Clinician-Administered PTSD scale (week 8).

All patents, patent applications, provisional applications, and publications referred to or cited herein are incorporated be reference in their entirety, including figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. A method of blunting, treating, or ameliorating trauma-related psychological disorders in an individual comprising the administration of a composition comprising one or more selective serotonin reuptake inhibitor (SSRI) compounds to an individual within 48 hours of a traumatic event.

2. The method according to claim 1, wherein said composition is administered within one (1) to 48 hours, 2 to 46 hours, 3 to 44 hours, 4 to 42 hours, 5 to. 40 hours, 6 to 38 hours, 7 to 36 hours, 8 to 34 hours, 9 to 32 hours, 10 to 30 hours, 11 to 28 hours, 12 to 24 hours, 12 to 26 hours, or 10 to 12 hours of the traumatic event.

3. The method according to claim 1, wherein said trauma-related psychological disorder is selected from the group consisting of acute stress disorder (ASD); post traumatic stress disorder (PTSD); depression; dysthymia; bipolar disorder; panic disorder; obsessive compulsive disorder (OCD), GAD; SAD; specific phobia; substance abuse/dependence; chronic pain; somatoform disorders; eating disorders; personality disorders; impulse control disorders; sleep disorders; and dissociative disorders.

4. The method according to claim 1, further comprising the continued administration of a composition comprising one or more SSRI compounds.

5. The method according to claim 1, wherein said SSRI are selected from the group consisting of: paroxetine; fluoxetine; sertraline; fluvoxamine; escitalopram; citalopram; combinations of up to six (6) of said SSRI; and salts, derivatives, or analogs thereof.

6. The method according to claim 1, wherein said individual is a human.

7. The method according to claim 1, wherein said individual is an animal.

8. The method according to claim 1, wherein said traumatic event is directly and personally experienced by the individual.

9. The method according to claim 1, wherein said traumatic event is witnessed by said individual.

10. The method according to claim 1, wherein said traumatic event comprises learning about a traumatic event experienced by a family member or close friend.

11. The method according to claims 1, 2, 3, 4, 5, 6, 7, 8, 9, or 10, wherein multiple doses of said composition are administered within 48 hours of said traumatic event.

12. A method of blunting, treating, or ameliorating trauma-related psychological disorders in an individual comprising the administration of a single dose of a composition comprising one or more SSRI compounds to an individual within 48 hours of a traumatic event.

13. The method according to claim 12, wherein said composition is administered within one (1) to 48 hours, 2 to 46 hours, 3 to 44 hours, 4 to 42 hours, 5 to 40 hours, 6 to 38 hours, 7 to 36 hours, 8 to 34 hours, 9 to 32 hours, 10 to 30 hours, 11 to 28 hours, 12 to 24 hours, 12 to 26 hours, or 10 to 12 hours of the traumatic event.

14. The method according to claim 12, wherein said trauma-related psychological disorder is selected from the group consisting of acute stress disorder (ASD); post traumatic stress disorder (PTSD); depression; dysthymia; bipolar disorder; panic disorder; obsessive compulsive disorder (OCD), GAD; SAD; specific phobia; substance abuse/dependence; chronic pain; somatoform disorders; eating disorders; personality disorders; impulse control disorders; sleep disorders; and dissociative disorders.

15. The method according to claim 12, wherein said SSRI are selected from the group consisting of: paroxetine; fluoxetine; sertraline; fluvoxamine; escitalopram; citalopram; combinations of up to six (6) of said SSRI; and salts, derivatives, or analogs thereof.

16. The method according to claim 12, wherein said individual is a human.

17. The method according to claim 12, wherein said individual is an animal.

18. The method according to claim 12, wherein said traumatic event is directly and personally experienced by the individual.

19. The method according to claim 12, wherein said traumatic event is witnessed by said individual.

20. The method according to claim 12, wherein said traumatic event comprises learning about a traumatic event experienced by a family member or close friend.
